(19) United States
(12) Patent Application Publication
(10) Pub. No.: US 2024/0350807 A1
(43) Pub. Date: Oct. 24, 2024

(54) SYSTEMS AND METHODS OF STIMULATION AT TRIGEMINALLY INNERVATED REGIONS FOR DISORDERS OF CEREBRAL PERFUSION

(71) Applicant: The Feinstein Institutes for Medical Research, Manhasset, NY (US)

(72) Inventors: Chunyan LI, Manhasset, NY (US); Timothy G. WHITE, Manhasset, NY (US); Kevin A. SHAH, Woodbury, NY (US); Henry WOO, Manhasset, NY (US)

(21) Appl. No.: 18/645,129

(22) Filed: Apr. 24, 2024

Related U.S. Application Data
(60) Provisional application No. 63/497,995, filed on Apr. 24, 2023.

(51) Int. Cl. A61N 1/36; A61N 1/04

(52) U.S. Cl. CPC A61N 1/36139; A61N 1/0456; A61N 1/36114

(57) ABSTRACT

Methods and apparatuses (e.g., systems) for stimulating trigeminal nerve with or without triggering an oxygen conserving reflex in a patient as a neuroprotective mechanisms when the patient is experiencing a decrease in cerebral perfusion due to a vascular injury and/or vascular degeneration. These methods and apparatuses may be configured to apply trigeminal stimulation to the patient in response to determining a need for cerebral perfusion improvement by the patient.

### CLAIM OF PRIORITY

This patent application claims priority to U.S. Provisional Patent Application No. 63/497,995, filed on Apr. 24, 2023, and titled “BIOELECTRIC THERAPY FOR ACUTE ISCHEMIC STOKE” which is herein incorporated by reference in its entirety.

### STATEMENT AS TO FEDERALLY SPONSORED RESEARCH

This invention was made with government support under W81XWH-18-1-0773 awarded by the Department of Defense, and NS114763 awarded by the National Institutes of Health. The government has certain rights in the invention.

### BACKGROUND

Impaired cerebral perfusion, such as that caused by Acute Ischemic stroke, results in a massive socioeconomic burden worldwide. Current therapies focus on achieving rapid reperfusion to prevent further ischemic injury. The main therapeutic interventions include fibrinolytic agents such as tissue plasminogen activator (such as alteplase or tenecteplase), or mechanical thrombectomy for reperfusion. While numerous non-fibrinolytic pharmacologic interventions have been tested for neuroprotective purposes, they have, to date, failed to demonstrate any benefit. The pharmacologic interventions are often single target agents that fail to translate from animal models to human benefit. Despite great successes of targeted pharmacotherapies in other fields, such as oncology, the continual failures of targeted neuroprotective agents in cerebral injury have led many investigators to support a pleiotropic mechanistic approach to cytoprotection. Bioelectronic medicine harnesses the body's intrinsic systems to deliver the sought-after pleiotropic end organ effects. There has been some inquiry into bioelectronic medicine in acute ischemic stroke but that has primarily focused on transcranial stimulation for disease recovery or vagal nerve stimulation for stroke rehabilitation. Interestingly, while the vagus nerve and facial nerve have been chosen as early modulation targets, their mechanistic targets are relatively narrow.

### SUMMARY OF THE DISCLOSURE

Described herein are methods and apparatuses (e.g., devices, systems, etc.) using trigeminal nerve stimulation (TNS) as a bioelectronic therapy for the treatment of acute ischemic stroke and other brain injuries with compromised cerebral perfusion. TNS has the potential to improve cerebral perfusion, protect the microvasculature, enhance cerebral resilience to injury, decrease neuroinflammation, and prevent harmful cortical spreading depolarizations, ultimately resulting in decreased neuronal injury and preservation of ischemic penumbral tissue. TNS can be applied to treat diseases of cerebral perfusion, particularly closed-loop TNS. The methods and apparatuses described herein, an in particularly the closed-loop TNS described herein may use the unique connections of the trigeminal nerve to induce cerebral vasodilation, induce peripheral vasoconstriction, decrease inflammation, decrease psychological dysfunction, increase wakefulness, protect neurovascular coupling, and modulate the blood-brain barrier. These methods and apparatuses may induce cerebral vasodilation in the micro- and macro-vasculatures to increase oxygen to injured tissue in the hyper-acute phase of traumatic conditions, preserving at-risk tissue. These methods and apparatuses may also modulate hypocretin production, which may regulate dopaminergic and glutamatergic signaling, in addition to the well-known effects on consciousness. This may decrease the development of brain damage, the benefits of which are compounded by the decrease of neuroinflammation. As such, closed-loop TNS may be impactful in the hyper-acute phase of primary brain injury, as well as conditions exhibiting secondary brain injury.

One form of bioelectronic medicine which can be applied to diseases of cerebral perfusion is trigeminal nerve stimulation (TNS) and, in particular, closed loop trigeminal nerve stimulation for activation of oxygen conserving reflexes. The unique connections of the trigeminal nerve allow its stimulation to induce cerebral vasodilation, induce peripheral vasoconstriction, decrease inflammation, decrease psychological dysfunction, increase wakefulness, protect neurovascular coupling, and modulate the blood-brain barrier. The induction of cerebral vasodilation in the micro- and macro-vasculatures increases oxygen to injured tissue in the hyper-acute phase of traumatic conditions, preserving at-risk tissue. Modulation of hypocretin production regulates dopaminergic and glutamatergic signaling, in addition to the well-known effects on consciousness. Therefore, closed loop TNS may be particularly impactful in the hyper-acute phase of disease where cerebral perfusion is impaired, as well as conditions exhibiting chronic derangements in cerebral perfusion. Closed-loop TNS has not yet been realized for actual therapeutic applications. The diving reflex is an endogenous oxygen conserving reflex mediated by the trigeminal nerve via closed loop feedback. In addition to the effects of TNS, which may be limited to the brain, it generates additional systemic effects, including bradycardia, systemic anti-oxidation and anti-inflammation, peripheral vasoconstriction, pulmonary vasodilation, splenic contraction, and angiogenesis. The diving reflex's unique connection between the brain and peripheral organs provides it the capacity to generate a unique suite of powerful systemic effects, both physiological and molecular, which are applicable to both the hyper-acute and chronic, rehabilitative, phases of stroke and other brain injuries with compromised cerebral perfusion. Electrical induction of diving reflex in humans has not been previously escribed or utilized for actual treatment.

The bioelectronic therapies described herein may improve patient care and provide acute ischemic stroke therapies, with the potential to reach a wide range of diseases, all stemming from disrupted cerebral perfusion. These methods and apparatuses may decrease the disability associated with disorders of cerebral perfusion, leading to a shortened rehabilitation periods for patients with cerebrovascular diseases and even a reduction in the number of individuals who end up requiring entrance to a rehabilitative care center in the first place. This could result in decreased healthcare resource utilization for these patients, improve patient outcomes, and redistribution of time and resources for treatment of other conditions.

Closed-loop TNS has not previously been realized for actual therapeutic applications. In general, the methods and apparatuses described herein, including the closed-loop TNS methods and apparatuses, may activate and/or monitor (e.g., receive feedback from) one or more cerebral perfusion reflexes, such as, but not limited to, one or more of: the diving reflex, the diving bradycardia, the diving response, the diving-induced hypoxia response, the oxygen-conserving reflex, the trigeminal reflex, and/or the trigeminocardiac reflex.

For example, the diving reflex is an endogenous oxygen conserving reflex mediated by the trigeminal nerve. In addition to the effects of TNS, which may be limited to the brain, it generates additional systemic effects, including bradycardia, systemic anti-oxidation and anti-inflammation, peripheral vasoconstriction, pulmonary vasodilation, splenic contraction, and angiogenesis. The diving reflex has been shown to increase cerebral perfusion, via cerebral vasodilation and peripheral vasoconstriction. Its unique connection to the brain and peripheral organs provides it the capacity to generate a unique suite of powerful systemic effects, both physiological and molecular, which is applicable to both the hyper-acute and chronic phases of stroke and other brain injuries with compromised cerebral perfusion.

The methods and apparatuses for bioelectronic therapies described herein may improve patient care, including providing acute ischemic stroke therapies, with the potential to reach a wide range of diseases related to disrupted cerebral perfusion including but not limited to subarachnoid hemorrhage, traumatic brain injury, vascular dementia, Alzheimer's disease and others. These methods and apparatuses may decrease the disability associated with disorders of cerebral perfusion, leading to a shortened rehabilitation periods for patients with cerebrovascular diseases and even a reduction in the number of individuals who end up requiring entrance to a rehabilitative care center in the first place. This could result in decreased healthcare resource utilization for these patients, improved patient outcomes, and redistribution of time and resources for treatment of other conditions. Stimulation of the trigeminal nerve causes not only parasympathetic activation, but also antidromic release of potent vasodilator peptides such as calcitonin gene-related peptide (CGRP) from the perivascular nerves, as well as intrinsic neuroprotective mechanisms via activation of brainstem nuclei. In addition to parasympathetic activation, TNS can activate the sympathetic nervous system via rostral ventrolateral medulla in the brainstem, resulting in peripheral vasoconstriction and an increase in blood pressure. In conjunction with cerebral vasodilation, caused by the vasodilatory neuropeptides, this results in a redistribution of blood to the central organs and an increase in cerebral perfusion. The trigeminal anatomical connections are believed to be the basis of oxygen conserving reflexes such as the diving reflex. The diving reflex can be induced by the apparatuses described herein using tuned, closed-loop TNS and may blunt ischemic injury. The methods and apparatuses described herein may be configured to use one or more reflexes (such as the diving reflex) as feedback for the closed-loop TNS. Such specifically tuned stimulation of the trigeminal nerve is a unique mechanism to improve perfusion to the central nervous system and provides great potential to prevent the progression of brain injuries of cerebral hypoperfusion by harnessing intrinsic neuroprotective mechanisms.

The methods and apparatuses (methods, systems, etc.) described herein may generally provide closed-loop electrical stimulation of the trigeminal nerve. These apparatuses may comprise a series of stimulus electrodes and sensor feedback devices communicatively coupled to the control device that may be configured to generate sensor data associated with the patient's status, and may include a control device communicatively coupled to the stimulus electrodes with pre-set programs for different disorders of cerebral perfusion. The stimulus electrodes described herein may be formed as patches with embedded electrodes. Sensor feedback devices may include a smart watch with blood pressure and heart rate monitors, a pulse oximeter to measure peripheral oxygen saturation levels and respiration rate, and the ability to communicate with other pre-existing sensors that assess cerebral perfusion. A control unit may induce the electrical stimulation of the trigeminal nerve. The control unit may receive inputs from the various sensory inputs and utilizing the input, tailor the stimulatory output to optimize cerebral perfusion. These methods and apparatuses may provide a closed-loop system for safe and reliable induction of the oxygen conserving reflex in the treatment of acute ischemic stroke as well as other diseases cerebral perfusion as previously mentioned.

Also described herein are stimulation parameters of trigeminal nerve that may provide TNS in various cerebrovascular diseases, such as stroke, traumatic brain injury, subarachnoid hemorrhage, and chronic cerebral hypoperfusion. The stimulation parameters may be general, and/or may be individually tuned based on the real-time monitored patient markers for safe and consistent delivering TNS to induce an oxygen conserving reflex (e.g., a diving reflex) as a treatment. The control unit may utilize pre-set programs based on sensor feedback to apply closed-loop stimulation patterns or tailor stimulation based on sensor feedback to deliver optimal individualized stimulation parameters.

For example, described herein are closed-loop methods for treating a disorder of cerebral perfusion, the method comprising: receiving sensor data associated with a patient's vascular status; determining, based on the sensor data, a need for improved cerebral perfusion by the patient; and activating or maintaining an oxygen conserving reflex in the patient by applying a non-invasive trigeminal stimulation to the patient's forehead.

A disorder of cerebral perfusion may generally include any disorder in which cerebral perfusion is reduced or compromised, including but not limited to stroke, subarachnoid hemorrhage, traumatic brain injury, vascular cognitive impairment, vascular dementia, and/or Alzheimer's disease. Activating or maintaining the oxygen conserving reflex may comprise applying the non-invasive trigeminal stimulation in a stimulation pattern that is closed-loop and targets the patient's trigeminal ophthalmic nerve branch, the patient's trigeminal maxillary nerve branch, or a combination thereof.

In some examples activating or maintaining the oxygen conserving reflex comprises applying the non-invasive trigeminal stimulation in a closed-loop manner in which a current or a voltage applied is titrated to increase the patient's systolic blood pressure by about 5-25 mmHg, decrease the patient's heart rate by about 5-10%, decrease the patient's peripheral oxygen saturation by about 5-20%, increase the patient's cerebral blood flow by about 5-30%. The trigeminal stimulation may be delivered for between 20 and 120 minutes after titration in a closed-loop manner. In some cases the loop (e.g., applying trigeminal stimulation during closed-loop treatment) may terminate automatically after a predetermined time (e.g., between 20 minutes and 4 hours, between 20 minutes and 3 hours, between 20 minutes and 2 hours, etc.), or it may be continued indefinitely.

The trigeminal stimulation may have parameters that include an intensity of between about 0.5 V and 45 V, with a pulse width between about 0.1 ms and 5 ms, a frequency between about 1 Hz and 200 Hz, and a duty cycle between about 0.5-30 seconds ON and about 1-30 minutes OFF, etc.

The sensor data may include heart rate data, blood pressure data, or a combination thereof. The sensor data may be analyzed by the controller and/or one or more processors to confirm that the patient is experiencing or maintaining the blood-conserving reflex (e.g., diving reflex) and may adjust the trigeminal stimulation accordingly. For example, the electrical stimulation of the subject's trigeminal nerve may be titrated by gradually increasing intensity to between about 0.5 V and 45 V by increasing by about 0.5 V per 30 seconds for more than about 5 minutes with pulse width between about 0.1 ms and 5 ms, frequency between about 1 Hz and 200 Hz, duty cycle between about 1-30 seconds ON and about 2-30 minutes OFF.

In some examples, the closed-loop stimulation activation may be based at least in part on periodic sampling of feedback markers including one or more of: the patient's heart rate, the patient's blood pressure, the patient's respiration rate, the patient's peripheral oxygen saturation, the patient's cerebral blood perfusion, the patient's pupillary response, and/or the patient's blood markers. In some examples determining the need for trigeminal nerve stimulation or further inducing oxygen conservation may be based on sensor data associated with decreased cerebral perfusion due to stroke, subarachnoid hemorrhage, traumatic brain injury, vascular cognitive impairment, vascular dementia, and/or Alzheimer's disease. As mentioned, activating or maintaining the oxygen conserving reflex may include inducing a diving reflex in the patient, and/or a trigeminocardiac reflex in the patient. Determining the need for oxygen conservation may be based on sensor data associated with stroke, subarachnoid hemorrhage, traumatic brain injury, vascular cognitive impairment, vascular dementia, and/or Alzheimer's disease. Activating the oxygen conserving reflex may include increasing a cerebral perfusion pressure.

Also described herein are systems for performing these methods. For example, described herein are systems for treating a disorder of cerebral perfusion in a patient, the system comprising: a sensor configured to generate sensor data associated with a patient's vascular status; a controller, communicatively coupled to the sensor, and configured to provide closed-loop stimulation based on data from the sensor indicating a need for oxygen conservation by the patient; and a stimulus electrode, communicatively coupled to the controller, and configured to deliver non-invasive trigeminal stimulation to the patient's trigeminal nerve from the controller to activate an oxygen conserving reflex in the patient.

The stimulus electrode may be included within a wearable patch configured to be worn on the patient's forehead. The controller may be integrated with the wearable patch. The controller may be configured to wirelessly receive the sensor data. The sensor data may include at least one of a patient's blood pressure and a patient's heart rate or other inputs from sensors that assess cerebral perfusion. The controller may be configured to implement a closed-loop feedback system by periodically sampling the sensor data and activating the oxygen conserving reflex based on the sensor data. The controller may be configured to increase cerebral perfusion pressure.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows an example of a transcutaneous trigeminal nerve stimulation system 100 to induce the oxygen conserving reflex.

FIG. 1B show an example of a transcutaneous trigeminal nerve stimulation system 100 to induce the oxygen conserving reflex.

FIG. 2 is a flowchart showing an example method 200 for activating a patient's oxygen conserving reflex.

FIG. 3 is a schematic illustration of an example oxygen related medical issues and a proposed apparatus and method to response to these medical issue.

FIG. 4 shows a block diagram of a controller that may be one example of the controller of FIG. 1A.

### DETAILED DESCRIPTION

The methods and apparatuses described herein provide closed-loop trigeminal nerve stimulation (TNS) as a bioelectronic therapy for the treatment of acute ischemic stroke and other brain injuries with compromised cerebral perfusion. Acute ischemic stroke is due to a vessel blockage from either a thrombus or an embolus. The fundamental pathophysiology of stroke is lack of blood supply leading to tissue death due to inability to maintain cellular metabolism. The ischemic penumbra is the viable tissue around the core infarct which is at risk of ischemia. As blood supply decreases, the ischemic lesion progresses and grows leading to infarction of penumbral tissue. Conventional therapies focus on achieving rapid reperfusion to the ischemic tissues to prevent further ischemic injury. Some therapeutic interventions include fibrinolytic agents (such as alteplase or tenecteplase), or mechanical thrombectomy for reperfusion. While numerous non-fibrinolytic pharmacologic interventions have been tested for neuroprotective purposes, they have failed to demonstrate any benefit.

Cerebral vasospasm (CV) and delayed cerebral ischemia (DCI) remain a leading cause of morbidity and mortality after aneurysmal subarachnoid hemorrhage (SAH). The pathogenesis of CV and DCI is complex, being related to microcirculatory derangements, loss of cerebral autoregulation, cortical spreading depolarizations, neuroinflammation, endothelial dysfunction, and micro thrombosis. However, the ultimate sequalae of DCI is new neurological injury and cerebral ischemia leading to worsening disability. Increasing perfusion to at risk territories has been associated with better outcomes in patients experiencing DCI and the gold standard medical therapy is to increase a person's blood pressure to enhance cerebral perfusion.

Dementia, including vascular dementia and Alzheimer's dementia, is one of the leading drivers of death and disability in developing countries. While numerous histopathological findings drove disease pathogenesis theories surrounding the development of plaques, research now suggests a role for impaired cerebral perfusion and neurovascular coupling as a driver of these diseases. Alzheimer's disease is the most common form of dementia, and a growing body of evidence has linked impaired neurovascular coupling and loss of perfusion to key areas of the brain to be a driver of this disease. Impaired perfusion leading to cognitive dysfunctional as well as metabolic mismatch results in slow neural degeneration and the buildup of plaques. These plaques then further strangle the cerebral microcirculation ultimately leading to cognitive decline and neuronal loss. Ultimately, evidence has been put forth demonstrating the vascular underpinning of numerous types of dementia. Thus, in some cases, dementia may be treated by the inducement of the oxygen conserving reflex thereby increasing cerebral perfusion.

The use of closed-loop trigeminal nerve stimulation may also decrease secondary injury in experimental models of traumatic brain injury by decreasing inflammation and ultimately decreasing lesional growth to prevent neurological worsening. In the setting of traumatic brain injury, trigeminal nerve stimulation can increase cerebral blood flow as well as restore cerebral autoregulation and neurovascular coupling.

Given the importance of hypoxia and ischemia for the treatment of cerebral perfusive diseases, the methods and apparatuses described herein may induce and/or use as feedback one or more oxygen conserving reflexes. Oxygen conserving reflexes, including cerebral perfusion reflexes, may include one or more of: the diving reflex, the diving bradycardia, the diving response, the diving-induced hypoxia response, the oxygen-conserving reflex, the trigeminal reflex, and/or the trigeminocardiac reflex. Conventional induction options for the diving reflex a form of an oxygen conserving reflex, such as ammonia vapors and cold-water facial immersion, are uncontrollable and unsuitable for treatment of critically ill patients; the methods described herein may electrically induce the diving reflex by tuned trigeminal electrical stimulation.

For example, an oxygen conserving reflex can be induced through an activation of the trigeminal nerve. Specifically tuned electronic stimulation of the trigeminal nerve can achieve a successful induction of the oxygen conserving reflex in a targeted and reproducible manner. The oxygen conserving reflex can be used to treat acute ischemic stroke, traumatic brain injury, subarachnoid hemorrhage, traumatic brain injury, vascular dementia, and Alzheimer's dementia. The oxygen conserving reflex can improve cerebral perfusion and restore neurovascular coupling. We further observed that targeting the trigeminal nerve stimulation to selectively induce the “oxygen conserving reflex”, was more effective as a treatment than merely performing general trigeminal nerve stimulation (limited to brain level).

The oxygen conserving reflex increases or improves cerebral perfusion. Decreased cerebral perfusion can be related to a lack of blood flow to a region of the brain or impaired cerebral autoregulation and/or neurovascular coupling, with the loss of the brain's ability to compensate hemodynamically. The trigeminal nerve may control cerebral blood flow by regulating both the cerebral macro and microcirculation and ultimately restoring neurovascular coupling.

The trigeminal nerve, the largest cranial nerve, provides sensory innervation of the face as well as innervating the arteries and arterioles up until the Virchow-Robinson space. The trigeminal nerve can increase cerebral blood flow through three distinct mechanisms: 1) antidromic projection of vasoactive peptides, 2) communication with the parasympathetic innervation of the cerebrovasculature, and 3) through intrinsic mechanisms acting at the microvasculature driven by communication with brainstem nuclei. These mechanisms can lead to potent vasodilation and improve cerebral perfusion, as well as restore neurovascular coupling with improved microcirculatory function. Trigeminal nerve stimulation has also been shown to induce pial arteriole vasodilation, and decrease microthrombi formation as well as induce small vessel vasodilation by nitric oxide dependent interneuron signaling. It can be titrated to controllably release multiple vasoactive molecules, including calcitonin gene related peptide (CGRP), pituitary adenylate cyclase-activating peptide (PACAP), nitric oxide (NO), substance P, adenosine triphosphate (ATP), and neurokinin A. In addition to producing vasoactive effects, these molecules produce numerous downstream effects including decreased oxidative stress, anti-inflammation, improved cell survival, and blood brain barrier modulation.

Trigeminal nerve stimulation can increase cerebral perfusion even in individuals with normal cerebral perfusion and intact autoregulation. Transcutaneous stimulation can stimulate the trigeminal nerve to volumetrically increase cerebral blood flow. For example, stimulation of the trigeminal nerve at 100 Hz with a variable voltage up to 32V for 20 minutes can provide a 10% increase in cerebral blood flow, at least in one example.

Trigeminal nerve stimulation can control various aspects of the neurovascular unit, including interneurons with the ability to intrinsically control cerebral blood flow, drive both endothelial dependent and independent vasodilation, pericytes/astrocytes with the ability to alter the bundle branch block, and microglia driving changes in the local immune profile, all ways to improve neurovascular coupling. Trigeminal nerve stimulation also leads to a decrease in inflammatory cytokines which have been shown to lower the threshold for cortical spreading depolarization induction and perpetuate neuronal injury.

The methods and apparatuses described herein may provide electrical trigeminal nerve stimulation-induced oxygen conserving reflex in various cerebrovascular diseases to ameliorate the adverse consequences of acute ischemic stroke, and also prevent and treat stroke induced vascular cognitive impairments and dementia.

For example, described herein are portable closed-loop trigeminal nerve stimulators that may include: a smart watch with blood pressure and heart rate monitor, a forehead auricle, side of the nasal bridge, and side of the eyebrows patch (e.g., single-use or multi-use) with embedded stimulation electrode, and an electronic stimulation unit. These methods and apparatuses may be used to treat acute stroke patients presenting with large vessel occlusion (e.g., ischemic stroke). In some examples, these methods may be used to treat one or more of: stroke, subarachnoid hemorrhage, traumatic brain injury, vascular cognitive impairment, vascular dementia, and/or Alzheimer's disease.

Any of the methods described herein may be used to activate a patient's oxygen conserving reflex. Any of the methods may include receiving sensor data associated with a patient's vascular status, determining based on the sensor data, a need for oxygen conservation by the patient, and activating an oxygen conserving reflex in the patient by applying non-invasive trigeminal stimulation to the patient's forehead, auricle, side of the eyebrows, underneath the eyes, and/or side of the nasal bridge.

In any of the methods described herein, the trigeminal stimulation can include stimulating the patient's trigeminal ophthalmic nerve branch, the patient's trigeminal maxillary nerve branch, the patient's remote trigeminal innervation, the patient's trigeminal auricular innervation, or a combination thereof. In any of the methods described herein, the non-invasive trigeminal stimulation may be performed by an electrode attached to the patient's forehead, auricle, side of the eyebrows, underneath the eyes, and/or side of the nasal bridge.

In any of the methods described herein, the sensor data can include heart rate data, blood pressure data, the patient's respiration rate, the patient's peripheral oxygen saturation, the patient's cerebral blood perfusion, the patient's pupillary response, the patient's blood markers or a combination thereof. In some examples, the sensor data can include other vital signs such as body temperature, galvanic responses, or the like.

In any of the methods described herein, TNS and/or activating the oxygen conserving reflex can be based at least in part on an open-loop system designed to provide treatment based on an initial titration to establish stimulation parameters.

In any of the methods described herein, TNS and/or activating the oxygen conserving reflex can be based at least in part on a closed-loop feedback system periodically sampling the sensor data and activating the oxygen conserving reflex based on the sensor data. In any of the methods described herein, determining the need for TNS and/or oxygen conservation may be based on sensor data associated with stroke, subarachnoid hemorrhage, traumatic brain injury, vascular cognitive impairment, vascular dementia, and/or Alzheimer's disease. In any of the methods described herein, activating the oxygen conserving reflex may include increasing cerebral perfusion pressure.

Any of the systems described herein may include a sensor configured to generate sensor data associated with a patient's vascular status, a controller, communicatively coupled to the sensor and configured to determine a need for oxygen conservation in the patient based on the sensor data, and a stimulus electrode, communicatively coupled to the controller and configured to provide trigeminal stimulation to the patient's trigeminal nerve with/without activating an oxygen conserving reflex in the patient.

In any of the systems described herein, the stimulus electrode may be included within a wearable patch configured to be worn on the patient's forehead. In some examples, the controller may be integrated with the wearable patch. In some other examples, the controller may be configured to wirelessly receive the sensor data.

In any of the system described herein, the sensor data can include at least one of a patient's blood pressure, a patient's heart rate the patient's respiration rate, the patient's peripheral oxygen saturation, the patient's cerebral blood perfusion, the patient's pupillary response, the patient's blood markers. In some examples, the controller may be configured to implement a closed-loop feedback system periodically sampling the sensor data and activating TNS and/or the oxygen conserving reflex based on the sensor data.

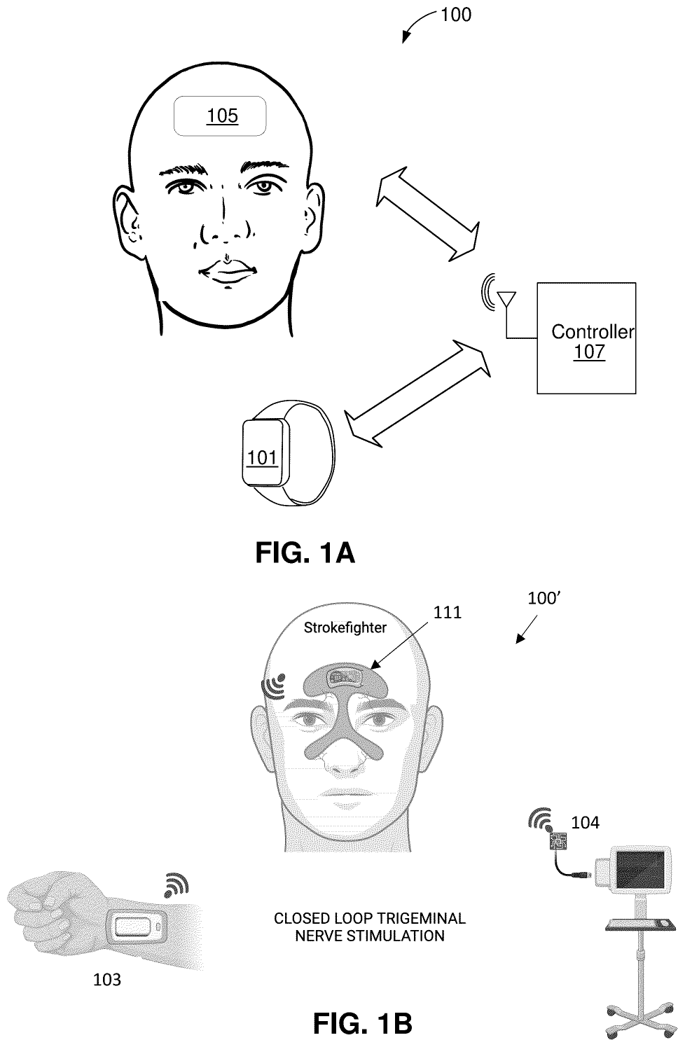

FIG. 1A shows an example of a transcutaneous trigeminal nerve stimulation system 100 to induce the oxygen conserving reflex. The system 100 may include a smart watch 101, a forehead electrode 105, and a controller 107. One example of an oxygen conserving reflex is the diving reflex. The diving reflex is a mammalian reflex that includes a set of physiological responses to water submersion. The diving reflex is believed to aid in the conservation of oxygen stores in mammals by initiating physiologic changes including bradycardia, apnea, and increased peripheral vascular resistance (peripheral vasocontraction). The diving reflex has been associated with increases in cerebral blood flow in humans with significant potential for neuroprotection by improving macro and microcirculatory perfusion. In some cases, the oxygen conserving reflex may be induced by stimulation of a trigeminal nerve through the forehead electrode 105.

The forehead electrode 105, which can be implemented as a forehead sticker, a wearable patch, or the like, may include one or more electrodes to stimulate the trigeminal nerve. In some examples, the forehead electrode 105 can include one or more ophthalmic stimulation electrodes. In general, the forehead electrode 105 may be positioned on the patient's forehead such that any included electrodes are positioned on or near the nasal bridge. In some examples, electrodes may be placed under the eyes (electrodes not shown). Generally, the forehead electrode 105 (more particularly electrodes included within the forehead electrode 105) may be used to deliver stimulation pulses (transdermal electrical stimulation) to the trigeminal nerve. In some examples, the forehead electrode 105 delivers stimulation pulses to a trigeminal ophthalmic nerve branch and or a trigeminal maxillary nerve branch of the trigeminal nerve.

The smart watch 101 may include one or more sensors for monitoring one or more patient bodily functions. Example monitored bodily functions include blood pressure and heart rate, however other monitored bodily functions are possible such, as blood oxygenation levels, body temperature, and the like. In general, sensor data from the smart watch 101 may be related to, or associated with, a patient's vascular status. Example smart watches may include an Apple Watch and an Android compatible watch; however any feasible wearable sensor may be used to provide sensor data. In some examples, the bodily function monitoring capability may be included within the forehead electrode 105.

The controller 107 can receive the sensor data (for example, from the smart watch 101 or from other sensors already in place on the subject) and cause the forehead electrode 105 to deliver electrical stimulation to the trigeminal nerve based on the sensor data. In some implementations, the controller 107 can interpret the sensor data including from external sensors to determine the likelihood that the patient is undergoing (or likely to undergo) a vascular injury (e.g., due to stroke, traumatic brain injury, subarachnoid hemorrhage, chronic cerebral hypoperfusion, etc.). In some other implementations, the controller 107 may determine whether the patient can benefit from the provocation of an oxygen conserving reflex.

FIG. 1B is another example of a transcutaneous trigeminal nerve stimulation system 100′ to induce the oxygen conserving reflex. In FIG. 1B, similar to that shown in FIG. 1A, the system 100′ may include a wearable component (e.g., smartwatch, etc.) 103, a forehead electrode 111, and a controller 104. As in FIG. 1A, the oxygen conserving reflex may be induced by stimulation of a trigeminal nerve through the forehead electrode 111.

As mentioned, the forehead electrode 111 can be implemented as a forehead sticker, a wearable patch, or the like, and may include one or more electrodes to stimulate the trigeminal nerve. The forehead electrode 111 may be positioned on the patient's forehead such that any included electrodes are positioned on or near the nasal bridge. In some examples, electrodes may be placed under the eyes (electrodes not shown). The forehead electrode 111 may be used to deliver stimulation pulses (transdermal electrical stimulation) to the trigeminal nerve, e.g., a trigeminal ophthalmic nerve branch and or a trigeminal maxillary nerve branch of the trigeminal nerve.

The wearable component 103 may include one or more sensors for monitoring one or more patient bodily functions. Example monitored bodily functions include blood pressure and heart rate, however other monitored bodily functions are possible such, as blood oxygenation levels, body temperature, and the like. Sensor data may be related to, or associated with, a patient's vascular status as well as the patient's cerebral perfusion from integrated external sensors.

The controller 104 can receive the sensor data and cause the forehead electrode 111 to deliver electrical stimulation to the trigeminal nerve based on the sensor data. In some implementations, the controller 104 can interpret the sensor data to determine the likelihood that the patient is undergoing (or likely to undergo) a vascular injury (e.g., due to stroke, traumatic brain injury, subarachnoid hemorrhage, chronic cerebral hypoperfusion, etc.). In some other implementations, the controller 104 may determine whether the patient can benefit from the provocation of an oxygen conserving reflex.

In some examples, the forehead electrode 105, 111 the wearable 101, 103 and the controller 107, 104 may be communicatively coupled together through any feasible wireless communication technology. Example wireless communication technologies can include those conforming to any of the IEEE 802.11 standards, Bluetooth protocols put forth by the Bluetooth Special Interest Group (SIG), Ethernet protocols near field communication (NFC) protocols, Zigbee protocols, or the like. In some cases, the forehead electrode 105, 111 the wearable 103 (e.g., smart watch 101), and the controller 107, 104 may be communicatively coupled together through any feasible wired communication protocol.

In some examples, the forehead electrode 105, 111 can include communication circuitry (wired and/or wireless communication circuitry), power supply circuitry, and electrical impulse generating circuitry. Thus, the controller 107, 104 can wirelessly communicate with the forehead electrode 105, 111 and cause the forehead electrode 105, 111 to generate and/or deliver electric stimulation pulses to the trigeminal nerve.

The controller 107, 104 can be a separate device (as shown in FIG. 1). In some examples, functionality of the controller 107 may be integrated with a smart phone or other external sensors of cerebral perfusion. In some other examples, the controller may be included within the wearable 103 (e.g., smart watch 101) or the forehead electrode.

The controller may be configured to operate as a closed-loop trigeminal nerve stimulator for controlling the application of treatment (stimulation) by the one or more electrodes on the forehead electrode. The controller can periodically evaluate sensor data (heart rate and/or blood pressure data) and determine an intensity (voltage) and frequency (hertz) of the electrical stimulation to apply to the forehead electrode to induce an oxygen conserving reflex. In some cases, the controller can evaluate sensor data to activate cerebral neuroprotection.

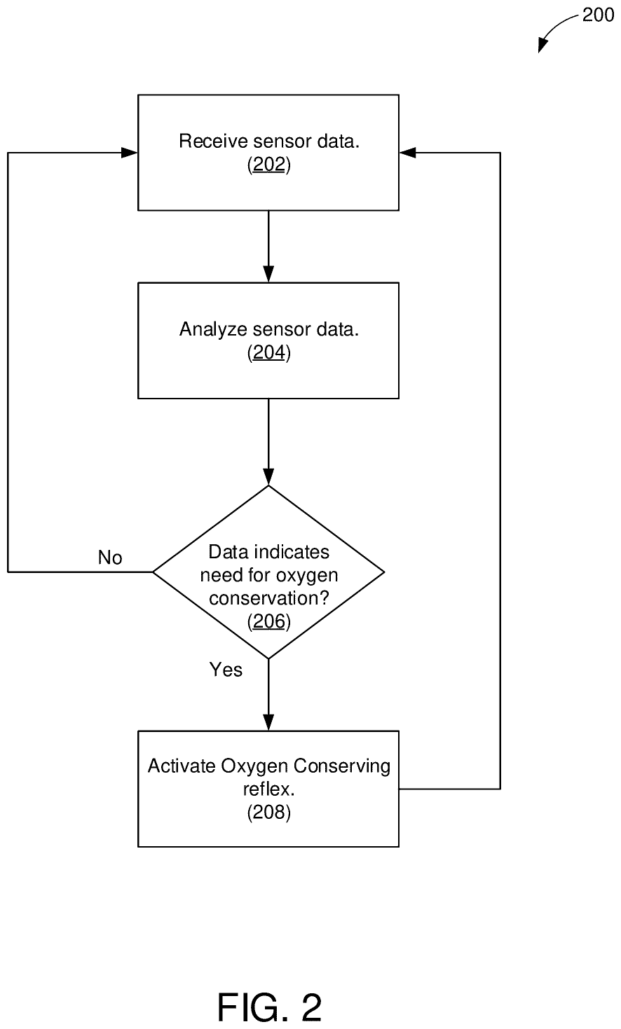

FIG. 2 is a flowchart showing an example method 200 for activating a patient's oxygen conserving reflex. Some examples may perform the operations described herein with additional operations, fewer operations, operations in a different order, operations in parallel, and some operations differently. The method 200 is described below with respect to the system 100 of FIG. 1, however, the method 200 may be performed by any other suitable system or device.

The method 200 begins in block 202 as the controller 107 receives sensor data. For example, the controller 107 can receive sensor data from the smart watch 101 or any other feasible sensor. In some cases, the controller 107 can receive sensor data from other various wearable sensors. In some examples, the sensor data includes heartbeat data and blood pressure data, although other data types are possible. In general, the sensor data may be related to or associated with a patient's vascular status as well as their cerebral perfusion.

The controller 107 may be communicatively coupled to the smart watch 101 or any other feasible sensor. For example, the controller 107 can be coupled to the smart watch 101 wirelessly or through a wire or cable. Thus, the controller 107 can receive sensor data through Wi-Fi, Bluetooth, or any other feasible wireless protocol.

Next, in block 204 the controller 107 analyzes the sensor data. In some examples, the controller 107 determines the need to activate an oxygen conserving reflex in a patient based on the sensor data. In some cases, the controller 107 can analyze the sensor data to determine if a vascular injury is present (such an injury may benefit from oxygen conservation). In some other examples, the controller 107 can determine if the sensor data indicates that characteristics of other illnesses or diseases are present such as, but not limited to, stoke, subarachnoid hemorrhage, traumatic brain injury, vascular cognitive impairment, vascular dementia, and/or Alzheimer's disease (such illnesses or diseases may also benefit from oxygen conservation).

Next, in block 206 the controller 107 determines if the sensor data indicates a need for oxygen conservation in the patient. If the sensor data does not indicate a need for oxygen conservation, then the method 200 returns to block 202. On the other hand, if the controller 107 determines that the sensor data indicates a need for oxygen conservation in the patient, then in block 208 the controller activates an oxygen conserving reflex in the patient. In some examples, the oxygen conserving reflex may be activated through non-invasive trigeminal stimulation to the patient's forehead. In some implementations, the trigeminal stimulation may be delivered through one or more patches placed on the patient's forehead. After the oxygen conserving reflex has been activated, the method returns to block 202.

In some examples, the return to block 202 can result in a periodic sampling and/or analysis of sensor data which can form a closed-loop feedback system. That is, the controller 107 can continuously (based on a sampling period) monitor sensor data and responsively activate an oxygen conserving reflex.

In some aspects, the oxygen conserving reflex may be used to increase cerebral perfusion pressure in the patient. In some other aspects, the non-invasive trigeminal stimulation may be used to induce a diving reflex in the patient. In some other examples, the oxygen conserving reflex can be any trigeminocardiac reflex.

Stimulation of the trigeminal nerve can occur on the subject's head (e.g., forehead). Stimulation parameters include a constant intensity (current or voltage, e.g., at between 0.5 V and 45 V more than 5 minutes) with pulse width between 0.1 ms and 5 ms, frequency between 1 Hz and 200 Hz, duty cycle between 0.5-30 seconds ON and 1-30 minutes OFF. Intermittent electrical stimulation of the subject's trigeminal nerve includes gradually increasing intensity (current or voltage, e.g., at between 0.5 V and 45 V by increasing more than 0.5 V per 30 seconds for more than 5 minutes) with pulse width between 0.1 ms and 5 ms, frequency between 1 Hz and 200 Hz, duty cycle between 1-30 seconds ON and 2-30 minutes OFF.

In some examples, the specific patient parameters can include an increase in the patient's systolic blood pressure by 5-10 mmHg, decrease in the patient's heart rate 5-10%, decrease in the patient's peripheral oxygen saturation 5-20%, increase in the patient's cerebral blood flow 5-20%, and/or decrease in the patient's respiration rate 10-30%.

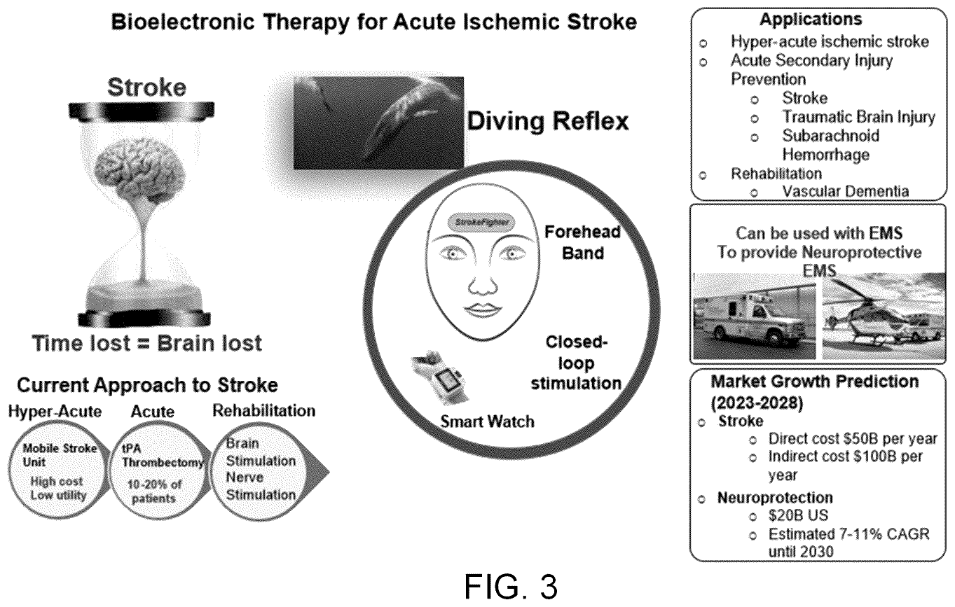

FIG. 3 is a schematic illustration of an example oxygen related medical issues and a proposed apparatus and method to response to these medical issue. FIG. 3 also shows example indications for use.

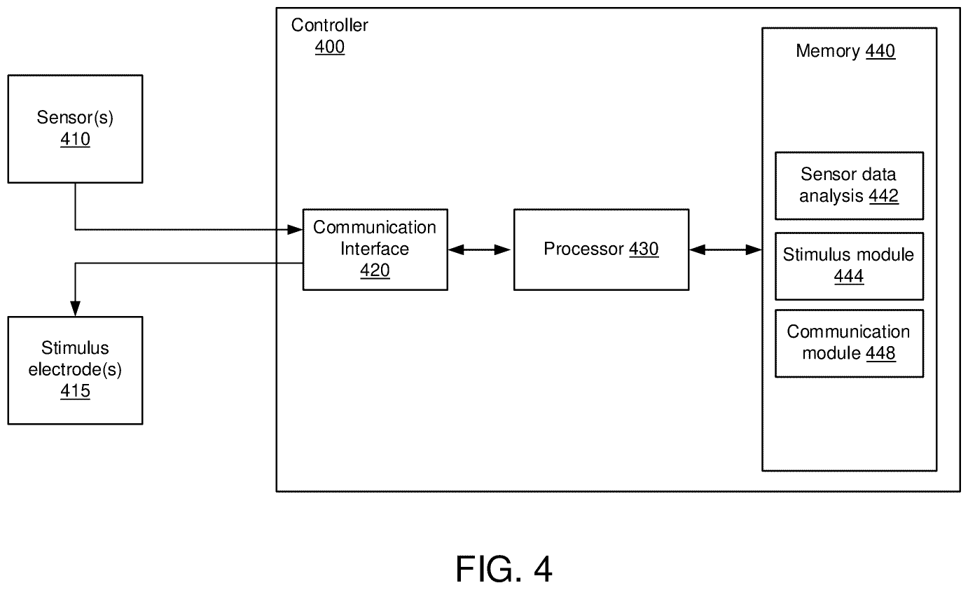

FIG. 4 shows a block diagram of a controller 400 that may be one example of the controller 107 of FIG. 1. The controller 400 may include a communication interface 420, a processor 430, and a memory 440.

The communication interface 420, which may be coupled one or more sensor(s) 410, one or more stimulus electrode(s) 415 and to the processor 430, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 420 may include wired (e.g., serial, ethernet, or the like) and/or wireless (Bluetooth, Wi-Fi, cellular, or the like) transceivers that may communicate with any other feasible device through any feasible network. In some examples, the communication interface 420 may receive sensor data from the sensor(s) 410 and may transmit commands to deliver trigeminal stimulation to a patient through the stimulus electrode(s) 415.

In some examples, the sensor(s) 410 can be another example of the smart watch 101 of FIG. 1. The sensor(s) 410 may provide heart rate and/or blood pressure data. In some other examples, the sensor(s) 410 can provide other patient data such as skin temperature, blood oxygen levels, galvanic responses and the like. In some examples the controller can assess data from other external sensors.

In some examples, the stimulus electrode(s) 415 can be another example of the electrodes 105. The stimulus electrode(s) 415 can provide non-invasive trigeminal stimulation to a patient's forehead to activate an oxygen conserving reflex, activate a diving reflex, increase cerebral perfusion pressure, or the like.

The processor 430, which is also coupled to communication interface 420 and the memory 440, may be any one or more suitable processors capable of executing scripts or instructions of one or more software programs stored in the controller 400 (such as within memory 440).

The memory 440 may include a non-transitory computer-readable storage medium (e.g., one or more nonvolatile memory elements, such as EPROM, EEPROM, Flash memory, a hard drive, etc.) that may store the following software modules: a sensor data analysis module 442 to analyze data from the sensor(s) 410; a stimulus module 444 to cause the stimulus electrode(s) 415 to provide non-invasive trigeminal stimulation; and a communication module 448 to communicate with the sensor(s) 410 and the stimulus electrode(s) 415.

Each software module includes program instructions that, when executed by the processor 430, may cause the controller 400 to perform the corresponding function(s). Thus, the non-transitory computer-readable storage medium of memory 440 may include instructions for performing all or a portion of the operations described herein.

The processor 430 may execute the sensor data analysis module 442 to receive sensor data, such as sensor data from the sensor(s) 410. In some examples, the sensor data analysis module 442 can receive heart rate sensor data, blood pressure data, or any other feasible patient data. Execution of the sensor data analysis module 442 may determine a need for oxygen conservation by the patient. In some examples, the sensor data analysis module 442 may determine if a vascular injury has occurred, or if oxygen conservation can be used to treat ischemic stroke, hemorrhagic shock, traumatic brain injury, subarachnoid hemorrhage, traumatic brain injury, vascular dementia, and/or Alzheimer's dementia. In some aspects, the sensor data analysis module 442 can determine if a diving reflex should be induced or if cerebral perfusion pressure should be increased.

The processor 430 may execute the stimulus module 444 to provide a non-invasive trigeminal nerve stimulation to a patient. Execution of the stimulus module 444 may cause the stimulus electrode(s) 415 to provide nerve stimulation to the patient.

The processor 430 may execute the communication module 448 to communicate with any other feasible devices. For example, execution of the communication module 448 may enable the controller 400 to communicate via cellular networks conforming to any of the LTE standards promulgated by the 3^{rd} Generation Partnership Project (3GPP) working group, Wi-Fi networks conforming to any of the IEEE 802.11 standards, Bluetooth protocols put forth by the Bluetooth Special Interest Group (SIG), Ethernet protocols, or the like. In some embodiments, execution of the communication module 448 may enable controller 400 to communicate with the sensor(s) 410 and/or the stimulus electrode(s) 415.

Thus, in any of these apparatuses and methods the one or more sensors may be configured to monitor and identify signals consistent with a vascular event or injury, on a continuous (or alternatively, discrete) manner. The apparatus and method may, upon detection of the vascular event or injury, cause the application of trigeminal stimulation sufficient to result in an OCR in the patient.

Thus, in any of these apparatuses and methods the one or more sensors may be configured to monitor and identify signals consistent with compromised cerebral perfusion, on a continuous (or alternatively, discrete) manner. The apparatus and method may, upon detection of the compromised cerebral perfusion, cause the application of trigeminal stimulation sufficient to result in a trigeminal nerve stimulation and/or oxygen conserving reflex in the patient.

In general, the apparatuses described herein may include one or more ophthalmic stimulation electrodes (e.g., embedded or integrated into a wearable forehead patch or sticker), and an electronic stimulation unit, to stimulate the trigeminal nerve and/or induce the oxygen conserving reflex in the patient. Any of these apparatuses may be adapted for use with one or more read outs from currently available blood pressure, heart rate, pulse oximetry, respiration rate, and/or cerebral perfusion monitors to control the stimulation provided by a TENS device implanted within a forehead, sticker.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A closed-loop method for treating a disorder of cerebral perfusion, the method comprising:
receiving sensor data associated with a patient's vascular status;
determining, based on the sensor data, a need for increased cerebral perfusion by the patient; and
activating or maintaining an oxygen conserving reflex in the patient by applying a non-invasive trigeminal stimulation to the patient's forehead.

2. The method of claim 1, wherein activating or maintaining the oxygen conserving reflex comprises applying the non-invasive trigeminal stimulation in a stimulation pattern that is closed-loop and targets the patient's trigeminal ophthalmic nerve branch, the patient's trigeminal maxillary nerve branch, or a combination thereof.

3. The method of claim 1, wherein activating or maintaining the oxygen conserving reflex comprises applying the non-invasive trigeminal stimulation in a closed-loop manner in which a current or a voltage applied is titrated to increase the patient's systolic blood pressure by 5-10 mmHg, decrease the patient's heart rate 5-10%, decrease the patient's peripheral oxygen saturation 5-20%, increase the patient's cerebral blood flow 5-20%, and/or decrease the patient's respiration rate 10-30%.

4. The method of claim 1 wherein the trigeminal stimulation is delivered for between 20 and 120 minutes after titration.

5. The method of claim 1, wherein the trigeminal stimulation has parameters that include an intensity of between 0.5 V and 45 V, with a pulse width between 0.1 ms and 5 ms, a frequency between 1 Hz and 200 Hz, and a duty cycle between 0.5-30 seconds ON and 1-30 minutes OFF.

6. The method of claim 1, wherein the sensor data includes heart rate data, blood pressure data, data on a patient's cerebral blood flow and/or perfusion, or a combination thereof.

7. The method of claim 1, wherein the electrical stimulation of the subject's trigeminal nerve is titrated by gradually increasing intensity to between 0.5 V and 45 V by increasing 0.5 V per 30 seconds for more than 5 minutes with pulse width between 0.1 ms and 5 ms, frequency between 1 Hz and 200 Hz, duty cycle between 1-30 seconds ON and 2-30 minutes OFF.

8. The method of claim 1, wherein the closed-loop stimulation activation is based at least in part on periodic sampling of feedback markers including one or more of: the patient's heart rate, the patient's blood pressure, the patient's respiration rate, the patient's peripheral oxygen saturation, the patient's cerebral blood perfusion, the patient's pupillary response, and/or the patient's blood markers.

9. The method of claim 1, wherein determining the need for trigeminal nerve stimulation or further inducing oxygen conservation is based on sensor data associated with decreased cerebral perfusion due to stroke, subarachnoid hemorrhage, traumatic brain injury, vascular cognitive impairment, vascular dementia, and/or Alzheimer's disease.

10. The method of claim 1, wherein activating or maintaining the oxygen conserving reflex includes inducing a diving reflex in the patient.

11. The method of claim 1, wherein activating or maintaining the oxygen conserving reflex includes inducing a trigeminocardiac reflex in the patient.

12. The method of claim 1, wherein determining the need for oxygen conservation is based on sensor data associated with stroke, subarachnoid hemorrhage, traumatic brain injury, vascular cognitive impairment, vascular dementia, and/or Alzheimer's disease.

13. The method of claim 1, wherein activating the oxygen conserving reflex includes increasing a cerebral perfusion pressure.

14. An apparatus for treating a disorder of cerebral perfusion in a patient, the apparatus comprising:
a sensor configured to generate sensor data associated with a patient's vascular status;
a controller, communicatively coupled to the sensor, and configured to provide closed-loop stimulation based on data from the sensor indicating a need for oxygen conservation by the patient; and
a stimulus electrode, communicatively coupled to the controller, and configured to deliver non-invasive trigeminal stimulation to the patient's trigeminal nerve from the controller to activate an oxygen conserving reflex in the patient.

15. The apparatus of claim 14, wherein the stimulus electrode is included within a wearable patch configured to be worn on the patient's forehead.

16. The apparatus of claim 15, wherein the controller is integrated with the wearable patch.

17. The apparatus of claim 14, wherein the controller is configured to wirelessly receive the sensor data.

18. The apparatus of claim 14, wherein the sensor data includes at least one of a patient's blood pressure and a patient's heart rate.

19. The apparatus of claim 14, wherein the controller is configured to implement a closed-loop feedback system by periodically sampling the sensor data and activating the oxygen conserving reflex based on the sensor data.

20. The apparatus of claim 14, wherein the stimulus electrode is configured to activate a diving reflex in the patient.

21. The apparatus of claim 14, wherein the stimulus electrode is configured to induce a trigeminocardiac reflex in the patient.

22. The apparatus of claim 14, wherein the controller is configured to increase cerebral perfusion pressure.

23. The apparatus of claim 14, wherein the non-invasive trigeminal stimulation includes stimulating the patient's trigeminal ophthalmic nerve branch, the patient's trigeminal maxillary nerve branch, or a combination thereof.
